(19) United States
(12) Patent Application Publication
(10) Pub. No.: US 2026/0272412 A1
(43) Pub. Date: Sep. 17, 2026

(54) ULTRASOUND PROBE, ULTRASONIC ENDOSCOPE, AND ULTRASOUND DIAGNOSTIC SYSTEM

(71) Applicant: FUJIFILM Corporation, Tokyo (JP)

(72) Inventor: Takatoshi HASE, Kanagawa (JP)

(73) Assignee: FUJIFILM Corporation, Tokyo (JP)

(21) Appl. No.: 19/558,362

(22) Filed: Mar. 5, 2026

(30) Foreign Application Priority Data
Mar. 13, 2025 (JP) 2025-040262

(51) Int. Cl. A61B 8/12; A61B 1/00; A61B 1/005; A61B 8/00; A61L 2/206; A61L 2/208; B06B 1/06

(52) U.S. Cl. CPC A61B 8/12; A61B 1/00087; A61B 1/0052; A61B 8/4455; A61L 2/206; A61L 2/208; B06B 1/0625

(57) ABSTRACT

Provided is an ultrasound probe that can suppress deterioration of a piezoelectric element caused by a gas used for sterilization.

An ultrasound probe includes: a support member that includes an outer peripheral surface having a cylindrical shape; a plurality of piezoelectric elements arranged on the outer peripheral surface in a circumferential direction of the outer peripheral surface; an acoustic lens that is disposed outside the plurality of piezoelectric elements in a radial direction of the outer peripheral surface and that covers the plurality of piezoelectric elements; and a gas barrier layer that is disposed between the plurality of piezoelectric elements and the acoustic lens, in which a permeation rate of a hydrogen peroxide gas or an ethylene oxide gas in the gas barrier layer is lower than a permeation rate of the hydrogen peroxide gas or the ethylene oxide gas in the acoustic lens, and, in the radial direction, the gas barrier layer is thinner than the acoustic lens.

### CROSS-REFERENCE TO RELATED APPLICATIONS

The present application claims priority under 35 U.S.C. § 119 to Japanese Patent Application No. 2025-040262, filed on Mar. 13, 2025. The above application is hereby expressly incorporated by reference, in its entirety, into the present application.

### BACKGROUND OF THE INVENTION

### 1. Field of the Invention

The present invention relates to an ultrasound probe, and more particularly, to an ultrasound probe that is used by being inserted into a subject. In addition, the present invention also relates to an ultrasonic endoscope and an ultrasound diagnostic system provided with the ultrasound probe.

### 2. Description of the Related Art

In the related art, for example, as disclosed in JP2018-064744A, a so-called radial-type ultrasound probe that is mounted on an insertion portion or the like of a so-called ultrasonic endoscope and that is used by being inserted into a subject is known. In order to generate an ultrasound image of an observation target located inside the subject, such an ultrasound probe performs transmission of an ultrasound beam toward the observation target and reception of an ultrasound echo from the observation target.

### SUMMARY OF THE INVENTION

Meanwhile, with respect to an ultrasound probe that is inserted into a subject, typically, sterilization using a gas such as an ethylene oxide gas or a hydrogen peroxide gas plasma is often performed after use for observation. In general, such an ultrasound probe disclosed in JP2018-064744A includes a piezoelectric element for transmitting and receiving ultrasound waves, and an acoustic lens that is bonded to the piezoelectric element with an adhesive or the like and that is formed of a material such as a silicone resin; however, a gas used for sterilization may permeate through the acoustic lens and the adhesive to reach the piezoelectric element, causing the piezoelectric element to deteriorate. In such a case, because of the deterioration, the piezoelectric element cannot sufficiently transmit and receive ultrasound waves in a desired frequency band, and, as a result, problems such as a decrease in resolution and sensitivity of the piezoelectric element may occur.

The present invention has been made in order to solve such problems in the related art, and an object thereof is to provide an ultrasound probe, an ultrasonic endoscope, and an ultrasound diagnostic system that can suppress deterioration of a piezoelectric element caused by a gas used for sterilization.

According to the following configuration, the above object can be achieved.
[1] An ultrasound probe comprising:
a support member that includes an outer peripheral surface having a cylindrical shape;
a plurality of piezoelectric elements arranged on the outer peripheral surface in a circumferential direction of the outer peripheral surface;
an acoustic lens that is disposed outside the plurality of piezoelectric elements in a radial direction of the outer peripheral surface and that covers the plurality of piezoelectric elements; and
a gas barrier layer that is disposed between the plurality of piezoelectric elements and the acoustic lens,
in which a permeation rate of a hydrogen peroxide gas or an ethylene oxide gas in the gas barrier layer is lower than a permeation rate of the hydrogen peroxide gas or the ethylene oxide gas in the acoustic lens, and
in the radial direction, the gas barrier layer is thinner than the acoustic lens.
[2] The ultrasound probe according to [1],
in which a ratio of a thickness of the gas barrier layer in the radial direction to a thickness of the acoustic lens in the radial direction is 1/15 or less.
[3] The ultrasound probe according to [1] or [2],
in which the piezoelectric element includes an acoustic matching layer, and
the gas barrier layer is disposed between the acoustic matching layer and the acoustic lens.
[4] The ultrasound probe according to any one of [1] to [3],
in which the plurality of piezoelectric elements are located inside the gas barrier layer in a direction along a center axis of the cylindrical shape of the support member.
[5] The ultrasound probe according to any one of [1] to [4],
in which an acoustic impedance of the gas barrier layer is smaller than an acoustic impedance of the piezoelectric element and is greater than an acoustic impedance of the acoustic lens.
[2] The ultrasound probe according to any one of [1] to [5],
in which the permeation rate of the hydrogen peroxide gas in the gas barrier layer is g/(m²·day) or less.
[2] The ultrasound probe according to any one of [1] to [6],
in which the gas barrier layer is a film wound around the plurality of piezoelectric elements.
[2] The ultrasound probe according to [7],
in which the gas barrier layer includes a connection region in which an adhesive that fills a space between both end portions of the gas barrier layer in the circumferential direction is located, or in which the gas barrier layer is overlapped in the circumferential direction.
[2] The ultrasound probe according to [8],
in which the connection region has, in the circumferential direction, a width dimension corresponding to an angular range greater than 0 degrees and equal to or less than 30 degrees about a center axis of the cylindrical shape of the support member.
[2] The ultrasound probe according to [8] or [9],
in which an overlapping portion of the gas barrier layer overlaps any of the plurality of piezoelectric elements.
[2] The ultrasound probe according to [8] or [9],
in which an overlapping portion of the gas barrier layer is located between a pair of piezoelectric elements adjacent to each other among the plurality of piezoelectric elements.
[2] The ultrasound probe according to [8],
in which the gas barrier layer is wound around the plurality of piezoelectric elements, with a gap formed in the connection region between both end portions of the gas barrier layer in an arrangement direction of the plurality of piezoelectric elements, and the gap is filled with a resin.
[2] The ultrasound probe according to [8],
in which the gas barrier layer includes a plurality of protrusions formed on a surface facing the plurality of piezoelectric elements and having a width narrower than an interval between a pair of piezoelectric elements adjacent to each other among the plurality of piezoelectric elements.
[2] The ultrasound probe according to [7],
in which the gas barrier layer has a seamless tubular shape.
[2] An ultrasonic endoscope comprising:
an endoscope insertion portion that is inserted into a body cavity of a subject and that extends in an axial direction; and
an endoscope observation portion and an ultrasound observation portion that are disposed at a distal end of the endoscope insertion portion,
in which the ultrasound observation portion includes the ultrasound probe according to any one of [1] to [14].
[2] The ultrasonic endoscope according to [15],
in which the gas barrier layer is a film wound around the plurality of piezoelectric elements along the circumferential direction of the outer peripheral surface, and has both end portions disposed together within a prescribed connection region, and
the connection region of the gas barrier layer is localized at a prescribed position in the circumferential direction of the outer peripheral surface.
[2] The ultrasonic endoscope according to [16],
in which the endoscope observation portion includes an optical observation window that, at a position eccentric with respect to a center axis of the endoscope insertion portion, is directed forward from the distal end of the endoscope insertion portion along the axial direction of the endoscope insertion portion, and
the connection region of the gas barrier layer is disposed on a side opposite to the optical observation window with respect to the center axis of the endoscope insertion portion.
[2] The ultrasonic endoscope according to [16], further comprising:
a treatment tool that is inserted through the endoscope insertion portion,
in which the endoscope observation portion includes a treatment tool outlet port that, at a position eccentric with respect to a center axis of the endoscope insertion portion, is directed forward from the distal end of the endoscope insertion portion along the axial direction of the endoscope insertion portion and through which the treatment tool is led out toward an inside of the subject, and
the connection region of the gas barrier layer is disposed on a treatment tool outlet port side with respect to the center axis of the endoscope insertion portion.
[2] The ultrasonic endoscope according to [16], further comprising:
an operation portion that is used by a user to operate the endoscope insertion portion,
in which the endoscope insertion portion includes a bending portion that bends in a prescribed upward direction in response to an operation of the user via the operation portion and in a downward direction opposite to the upward direction, and
the connection region of the gas barrier layer is disposed on a downward direction side with respect to a center axis of the endoscope insertion portion.
[2] An ultrasound diagnostic system comprising:
the ultrasonic endoscope according to [16]; and
an ultrasound processor device connected to the ultrasonic endoscope,
in which the ultrasound processor device generates an ultrasound image based on a reception signal obtained by transmitting and receiving ultrasound waves toward a subject via the ultrasound observation portion,
the ultrasound image has an upper end portion, a lower end portion, a probe region corresponding to the ultrasound probe, and a tomographic region outside the probe region,
a plurality of positions on a contour of the probe region correspond to the plurality of piezoelectric elements of the ultrasound probe, and
the connection region of the gas barrier layer is disposed, in the circumferential direction of the outer peripheral surface, at a position on the contour of the probe region in the ultrasound image that corresponds to a position on an upper end portion side.

In the present invention, the ultrasound probe comprises: a support member that includes an outer peripheral surface having a cylindrical shape; a plurality of piezoelectric elements arranged on the outer peripheral surface in a circumferential direction of the outer peripheral surface; an acoustic lens that is disposed outside the plurality of piezoelectric elements in a radial direction of the outer peripheral surface and that covers the plurality of piezoelectric elements; and a gas barrier layer that is disposed between the plurality of piezoelectric elements and the acoustic lens, in which a permeation rate of a hydrogen peroxide gas or an ethylene oxide gas in the gas barrier layer is lower than a permeation rate of the hydrogen peroxide gas or the ethylene oxide gas in the acoustic lens, and, in the radial direction, the gas barrier layer is thinner than the acoustic lens. Therefore, deterioration of the piezoelectric element caused by a gas used for sterilization can be suppressed.

### BRIEF DESCRIPTION OF THE DRAWINGS

FIG. 1 is a diagram showing a configuration of an ultrasound diagnostic system in an embodiment of the present invention.

FIG. 2 is an enlarged view of a distal end portion of an insertion portion of an ultrasonic endoscope provided with an ultrasound probe of the embodiment of the present invention.

FIG. 3 is a diagram showing the ultrasound probe of the embodiment of the present invention.

FIG. 4 is a diagram showing a laminated structure of the ultrasound probe of the embodiment of the present invention.

FIG. 5 is a diagram schematically showing a state in which a film-shaped gas barrier layer is wound around a plurality of piezoelectric elements in the embodiment of the present invention.

FIG. 6 is a diagram showing an example of the gas barrier layer wound over the plurality of piezoelectric elements in the embodiment of the present invention.

FIG. 7 is a diagram showing a first example of an overlapping portion between one end and the other end of the film-shaped gas barrier layer wound over the plurality of piezoelectric elements.

FIG. 8 is a diagram showing a second example of the overlapping portion between the one end and the other end of the film-shaped gas barrier layer wound over the plurality of piezoelectric elements.

FIG. 9 is a diagram schematically showing an example in which a resin fills a space between the one end and the other end of the film-shaped gas barrier layer wound over the plurality of piezoelectric elements.

FIG. 10 is a diagram showing an example of an overlapping portion between one end and the other end of a gas barrier layer in a first modification example wound over the plurality of piezoelectric elements.

FIG. 11 is a cross-sectional view of the gas barrier layer in the first modification example.

FIG. 12 is a diagram showing an example of an overlapping portion between one end and the other end of a gas barrier layer in a second modification example wound over the plurality of piezoelectric elements.

FIG. 13 is a diagram showing a gas barrier layer in a third modification example wound over the plurality of piezoelectric elements.

FIG. 14 is a diagram showing a gas barrier layer in a fourth modification example having a tubular shape.

FIG. 15 is a diagram showing a positional relationship of a connection region in the ultrasonic endoscope of the embodiment of the present invention.

FIG. 16 is a diagram showing an example of an ultrasound image captured by the ultrasonic endoscope of the embodiment of the present invention.

### DESCRIPTION OF THE PREFERRED EMBODIMENTS

Hereinafter, embodiments of the invention will be described with reference to the accompanying drawings.

The descriptions of configuration requirements to be described below are based on representative embodiments of the present invention, but the present invention is not limited to such embodiments.

Note that, in the present specification, a numerical range represented using “~” means a range including, as lower-limit and upper-limit values, the numerical values to be described before and after “~”.

In the present specification, “identical” and “same” are construed to include an error range generally allowable in the technical field.

### Embodiment

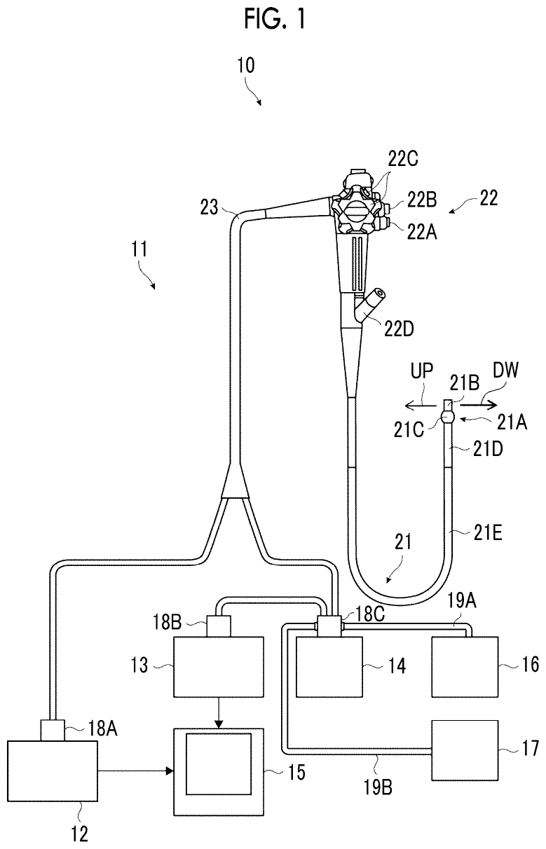

FIG. 1 shows a configuration of an ultrasound diagnostic system 10 in an embodiment of the present invention. The ultrasound diagnostic system 10 comprises an ultrasonic endoscope 11, an ultrasound processor device 12, an endoscope processor device 13, a light source device 14, a monitor 15, a water supply tank 16, and a suction pump 17.

The ultrasound processor device 12 is connected to the ultrasonic endoscope 11 via an ultrasound connector 18A. The endoscope processor device 13 is connected to the ultrasonic endoscope 11 via an endoscope connector 18B. The monitor 15 is connected to the ultrasound processor device 12 and the endoscope processor device 13. The light source device 14 is connected to the ultrasonic endoscope 11 and the endoscope processor device 13 via a light source connector 18C. Additionally, the water supply tank 16 is connected to the light source connector 18C via an air/water supply tube 19A, and the suction pump 17 is connected to the light source connector 18C via a suction tube 19B.

The ultrasonic endoscope 11 includes an endoscope insertion portion 21 that is inserted into a subject from an oral cavity or the like of the subject and that extends in an axial direction, an operation portion 22 used by a user such as a physician to operate the ultrasonic endoscope 11, and a universal cord 23 connected to the ultrasound processor device 12, the endoscope processor device 13, the light source device 14, the water supply tank 16, and the suction pump 17.

The endoscope insertion portion 21 includes, in order from a distal end side, a distal end portion 21A formed of a rigid member and including an endoscope observation portion 21B and an ultrasound observation portion 21C, a bending portion 21D coupled to a proximal end side of the distal end portion 21A, and a flexible portion 21E that couples a proximal end side of the bending portion 21D and a distal end side of the operation portion 22 to each other. The endoscope observation portion 21B includes a so-called solid-state imaging element, and acquires an captured image signal obtained by photoelectrically converting reflected light from an observation target in a body cavity of the subject. The ultrasound observation portion 21C includes a so-called ultrasound oscillator, and acquires a reception signal obtained by converting a so-called ultrasound echo from the observation target in the subject into an electrical signal. The bending portion 21D is configured by coupling a plurality of bending pieces (angulation rings) and bends in accordance with an operation of the user via the operation portion 22. The flexible portion 21E has an elongated and long shape and has flexibility. In addition, the ultrasonic endoscope 11 includes a treatment tool, such as forceps, that is inserted through the endoscope insertion portion 21, and inside the endoscope insertion portion 21, an air and water supply conduit, a suction conduit, and, as will be described below, a treatment tool channel through which the treatment tool inserted from the operation portion 22 passes are formed.

The operation portion 22 is a member used by the user to operate the endoscope insertion portion 21, and includes an air/water supply button 22A, a suction button 22B, an angulation knob 22C, and a treatment tool insertion port 22D. Inside the operation portion 22, an air and water supply conduit, a suction conduit, and a treatment tool channel are formed, and these are respectively connected to the air and water supply conduit, the suction conduit, and the treatment tool channel of the endoscope insertion portion 21.

The air/water supply button 22A is a button that the user operates to supply water stored in the water supply tank 16 from the distal end portion 21A of the endoscope insertion portion 21 into the subject and to supply air from the distal end portion 21A of the endoscope insertion portion 21 into the subject. The suction button 22B is a button that the user operates to suction blood and the like present in the subject at the distal end portion 21A of the endoscope insertion portion 21. The angulation knob 22C is a member that the user operates to bend the bending portion 21D of the endoscope insertion portion 21. The bending portion 21D bends toward each of a prescribed upward direction UP and a downward direction DW opposite to the upward direction UP, in response to the operation of the user via the angulation knob 22C. The treatment tool insertion port 22D is an opening portion through which the treatment tool, such as forceps, is inserted.

The universal cord 23 includes a plurality of wiring cables through which the ultrasonic endoscope 11, and the ultrasound processor device 12 and the endoscope processor device 13 are electrically connected to each other; a light guide such as an optical fiber through which the ultrasonic endoscope 11 and the light source device 14 are optically connected to each other; an air and water supply conduit through which the ultrasonic endoscope 11 and the light source connector 18C are connected to each other; and a suction conduit through which the ultrasonic endoscope 11 and the light source connector 18C are connected to each other.

The ultrasound processor device 12 transmits a drive signal representing a transmission pattern of ultrasound waves to a plurality of ultrasound oscillators, which will be described below, provided in the ultrasound observation portion 21C, thereby causing the plurality of ultrasound oscillators to generate ultrasound waves. Additionally, the ultrasound processor device 12 generates an ultrasound image representing a tomographic image of the inside of the subject by processing the reception signal generated by converting the ultrasound echo in the ultrasound observation portion 21C.

The ultrasound processor device 12 amplifies a plurality of reception signals in an analog format received from the ultrasound observation portion 21C, and then converts the plurality of reception signals into a plurality of pieces of reception data in a digital format. The ultrasound processor device 12 performs so-called receive focus processing by imparting respective delays to the plurality of pieces of reception data and adding the plurality of pieces of delayed reception data. By this receive focus processing, sound-line signals are acquired in which each piece of reception data is added in phase and the focus of the ultrasound echo is narrowed. The ultrasound processor device 12 performs, on the sound-line signals, correction for attenuation with a distance according to the depth of the reflection position of the ultrasound wave using a preset sound speed value, and then performs envelope detection processing, thereby generating a B-mode image signal that is tomographic image information regarding tissue in the subject. The ultrasound processor device 12 converts the B-mode image signal into an image signal that follows a scanning method of a normal television signal (raster conversion), and performs various necessary image processing such as gradation processing on the converted B-mode image signal. Hereinafter, the B-mode image signal generated in this manner will be referred to as an ultrasound image.

The endoscope processor device 13 receives a captured image signal (data) acquired at the endoscope observation portion 21B from a site of the observation target illuminated with illumination light from the light source device 14, and performs various kinds of signal (data) processing and image processing on the received captured image signal, thereby generating an endoscope image that is an optical image.

In the present embodiment, respective processes in the ultrasound processor device 12 and the endoscope processor device 13 are executed by any computer. In addition, any computer may execute these processes by means of a processor as hardware, a program as software, or a combination thereof. In that case, the processor, in cooperation with the program, may be configured to execute various processes in the present embodiment and function as each unit or each means in the present embodiment. Further, the execution order of the processes by the processor is not limited to the order described and may be changed as appropriate. Any computer may be a general-purpose computer, a computer for a specific application, a workstation, or another system configured to execute the respective processes.

The processor may be configured using one or a plurality of pieces of hardware, and the type of hardware is not limited. For example, the processor may be configured using hardware such as a central processing unit (CPU), a micro-processing unit (MPU), a programmable logic device such as a field-programmable gate array (FPGA), a dedicated circuit for executing specific processing, such as an application-specific integrated circuit (ASIC), a graphics processing unit (GPU), or a neural processing unit (NPU). Additionally, the type of hardware may be a combination of different types of hardware. In a case where a plurality of pieces of hardware are configured to execute one or a plurality of processes of the processor, the plurality of pieces of hardware may be present in devices physically separate from each other or may be present in the same device. Further, in any embodiment, the order of each process by the processor is not limited to the order described above and may be changed as appropriate. Note that the hardware is configured using an electrical circuit (circuitry) or the like formed by a combination of circuit elements such as semiconductor elements.

Furthermore, the program may be software such as firmware or microcode. The program may also be, for example, a group of program modules, and each function thereof may be implemented by a processor configured to execute the corresponding function. The program may be a program code or a plurality of code segments stored in one or a plurality of non-transitory computer-readable media (for example, a storage medium or other storage, or the like). The program may be stored in a distributed manner across a plurality of non-transitory computer-readable media that are present in devices physically separate from each other. The program code or code segments may represent procedures, functions, subprograms, routines, subroutines, modules, software packages, classes, or any combination of instructions, data structures, or program statements. The program code or code segments may be connected to other code segments or to hardware circuits by transmitting and receiving information, data, arguments, parameters, or contents of memory.

The light source device 14 generates illumination light for causing the endoscope observation portion 21B to image the site of the observation target in the subject and to acquire the captured image signal, the illumination light being white light consisting of three primary colors of light, namely red light (R), green light (G), and blue light (B), or light having a specific wavelength. Illumination light generated in the light source device 14 propagates through the light guide in the ultrasonic endoscope 11 and is emitted from the endoscope observation portion 21B into the subject. The light source device 14 includes, for example, a light source such as a light-emitting diode (LED), a xenon lamp, a halogen lamp, and a fluorescent lamp.

The monitor 15 displays the ultrasound image generated by the ultrasound processor device 12, the endoscope image generated by the endoscope processor device 13, and the like. The monitor 15 includes, for example, a display device such as a liquid-crystal display (LCD) or an organic electroluminescence (EL) display.

The water supply tank 16 is a tank that stores degassed water, and is connected to the light source connector 18C via the air/water supply tube 19A. The degassed water from the water supply tank 16 is used as a cleaning liquid that is ejected from the endoscope observation portion 21B toward a surface of the endoscope observation portion 21B after passing through the air/water supply tube 19A, the light source connector 18C, the universal cord 23, the air and water supply conduit inside the operation portion 22, and the air and water supply conduit inside the endoscope insertion portion 21. The ultrasound diagnostic system 10 can further comprise, in addition to the water supply tank 16, an air supply pump that supplies air, from the endoscope observation portion 21B, to a predetermined air-supply destination, and the like.

The suction pump 17 is connected to the light source connector 18C via the suction tube 19B. The suction pump 17 is in communication with the universal cord 23, the suction conduit inside the operation portion 22, and the suction conduit inside the endoscope insertion portion 21, via the suction tube 19B and the light source connector 18C, and is used to suction, from the endoscope observation portion 21B, matter to be suctioned in the body cavity (including the degassed water supplied for cleaning).

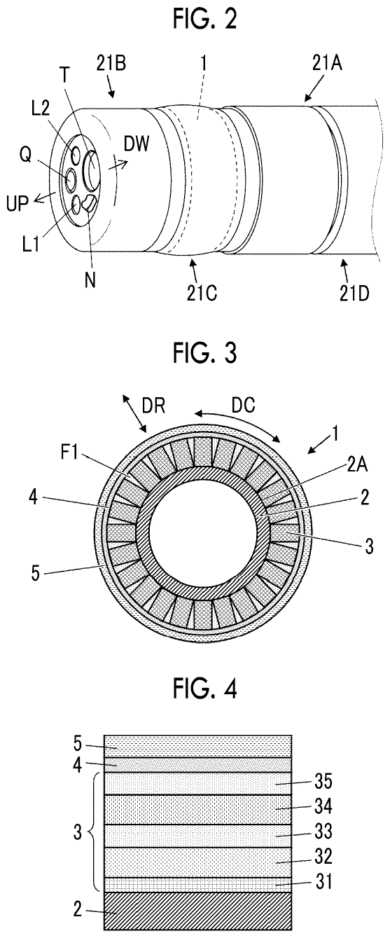

FIG. 2 shows an enlarged view of the distal end portion 21A in the ultrasonic endoscope 11. In the distal end portion 21A, the ultrasound observation portion 21C is located on a bending portion 21D side relative to the endoscope observation portion 21B.

The endoscope observation portion 21B includes, at the distal end thereof, an optical observation window Q, a treatment tool outlet port T, a first illumination window L1, a second illumination window L2, and a cleaning nozzle N. In addition, the endoscope observation portion 21B includes, inside the optical observation window Q, an objective lens (not shown) and a solid-state imaging element (not shown).

The first illumination window L1 and the second illumination window L2 are disposed on both sides of the optical observation window Q. An emission end of the light guide inside the ultrasonic endoscope 11 is connected to the first illumination window L1 and the second illumination window L2. Therefore, light emitted from the light source device 14 propagates through the light guide inside the ultrasonic endoscope 11 and is emitted into the body cavity of the subject from the first illumination window L1 and the second illumination window L2.

The optical observation window Q is attached at a position eccentric with respect to a longitudinal axial direction of the endoscope insertion portion 21, that is, with respect to a center axis of the endoscope insertion portion 21, in a state of being directed forward from the distal end of the endoscope insertion portion 21 along the axial direction of the endoscope insertion portion 21. Light emitted from the first illumination window L1 and the second illumination window L2 to the observation target in the body cavity of the subject is reflected by the observation target. The reflected light is incident on the optical observation window Q, passes through the objective lens, and is imaged on an imaging surface of the solid-state imaging element.

The solid-state imaging element outputs the captured image signal by photoelectrically converting reflected light from a site adjacent to the observation target, which has been imaged on the imaging surface after passing through the optical observation window Q and the objective lens. As the solid-state imaging element, for example, a charge-coupled device (CCD), a complementary metal-oxide-semiconductor (CMOS), and the like can be used. The captured image signal output from the solid-state imaging element is transmitted to the endoscope processor device 13 by the universal cord 23 via wiring cables provided in the endoscope insertion portion 21 and the operation portion 22. The endoscope image is generated by processing the captured image signal in the endoscope processor device 13.

Here, the upward direction UP, which is one of directions in which the bending portion 21D of the endoscope insertion portion 21 bends, can also be said to be a direction that the optical observation window Q faces with respect to the center axis of the endoscope insertion portion 21. As will be described below, the upward direction UP corresponds, in the ultrasound image generated in the ultrasound observation portion 21C, to a direction from an upper end toward a lower end.

The treatment tool outlet port T is an opening that, at a position eccentric with respect to the center axis of the endoscope insertion portion 21, is directed forward from the distal end of the endoscope insertion portion 21 along the axial direction of the endoscope insertion portion 21 and through which the treatment tool, such as forceps, that is inserted from the treatment tool insertion port 22D is led out toward the inside of the subject. The treatment tool outlet port T is disposed on a side opposite to the optical observation window Q with respect to the center axis of the endoscope insertion portion 21, that is, on a downward direction DW side relative to the optical observation window Q.

The cleaning nozzle N is disposed between the first illumination window L1 and the treatment tool outlet port T and is connected to an end portion of the air and water supply conduit inside the endoscope insertion portion 21. The cleaning nozzle N ejects, for cleaning, degassed water supplied from the water supply tank 16 onto the surfaces of the optical observation window Q, the first illumination window L1, and the second illumination window L2.

The ultrasound observation portion 21C comprises an ultrasound probe 1 of the embodiment of the present invention, which has a cylindrical shape. The ultrasound probe 1 is used to transmit and receive ultrasound waves to and from the observation target in the subject in a state of being inserted into the subject. The ultrasound probe 1 converts the ultrasound echo from the observation target into a reception signal, which is an electrical signal, and the reception signal is converted into the ultrasound image by an image processing device (not shown).

FIG. 3 shows a cross-section of the ultrasound probe 1 taken along a plane perpendicular to a direction along the center axis of the endoscope insertion portion 21. The ultrasound probe 1 comprises: a support member 2 including an outer peripheral surface 2A having a cylindrical shape; a plurality of piezoelectric elements 3 annularly arranged over an entire circumference in a circumferential direction DC of the outer peripheral surface 2A of the support member 2; an annular acoustic lens 5 that is disposed outside the plurality of piezoelectric elements 3 in a radial direction DR of the outer peripheral surface 2A of the support member 2 and that covers the plurality of piezoelectric elements 3; and a gas barrier layer 4 disposed between the plurality of piezoelectric elements 3 and the acoustic lens 5. A space between the plurality of piezoelectric elements 3 is filled with a resin F1 such as a silicone resin. Here, the circumferential direction DC of the outer peripheral surface 2A of the support member 2 refers to a direction along the outer peripheral surface 2A of the support member 2 on the plane perpendicular to the center axis of the cylindrical shape of the support member 2, and the radial direction DR of the outer peripheral surface 2A of the support member 2 refers to a direction from a center of the outer peripheral surface 2A of the support member 2 toward the outer peripheral surface 2A on the plane perpendicular to the center axis of the cylindrical shape of the support member 2. The circumferential direction DC and the radial direction DR of the outer peripheral surface 2A are orthogonal to each other.

The support member 2 is a member for supporting the plurality of piezoelectric elements 3, and is formed of, for example, a metal such as stainless steel.

As shown in FIG. 4, the piezoelectric element 3 includes a flexible printed wiring board 31 disposed on the outer peripheral surface of the support member 2, a backing material layer 32 disposed on the flexible printed wiring board 31, a dematching layer 33 disposed on the backing material layer 32, an ultrasound oscillator 34 disposed on the dematching layer 33, and an acoustic matching layer 35 disposed on the ultrasound oscillator 34. The gas barrier layer 4 is disposed on the acoustic matching layer 35, and the acoustic lens 5 is disposed on the gas barrier layer 4. That is, the gas barrier layer 4 is disposed between the acoustic matching layer 35 and the acoustic lens 5.

The ultrasound oscillator 34 transmits ultrasound waves in accordance with a prescribed drive signal and receives ultrasound echoes from the subject to output a reception signal based on the ultrasound echoes. The ultrasound oscillator 34 is configured by forming electrodes on both ends of a piezoelectric body consisting of, for example, a piezoelectric ceramic typified by lead zirconate titanate (PZT), a polymer piezoelectric element typified by polyvinylidene difluoride (PVDF), a piezoelectric single crystal typified by lead magnesium niobate-lead titanate (PMN-PT), or the like.

The acoustic matching layer 35 matches an acoustic impedance between the ultrasound oscillator 34 and the subject that is in contact through the acoustic lens 5, to facilitate incidence of ultrasound waves on the subject. The acoustic matching layer 35 can be formed of a material having an acoustic impedance smaller than an acoustic impedance of the ultrasound oscillator 34 and greater than an acoustic impedance of the subject, and can be formed of, for example, an epoxy-based resin, a silicone resin, a polyimide resin, a polyethylene resin, or the like.

The dematching layer 33 is formed of a material having an acoustic impedance greater than the acoustic impedance of the ultrasound oscillator 34, and reflects, toward a subject side, ultrasound waves propagating in a direction from the ultrasound oscillator 34 toward the backing material layer 32. The dematching layer 33 is mainly formed of, for example, a urethane resin, an epoxy resin, a silicone resin, or the like.

The backing material layer 32 supports the dematching layer 33 and the ultrasound oscillator 34. In addition, the backing material layer 32 attenuates ultrasound waves that propagate to the backing material layer 32 among ultrasound waves emitted from the ultrasound oscillator 34 and ultrasound echoes reflected from the observation target. The backing material layer 32 is formed of a material having rigidity equal to or greater than a certain level, such as hard rubber, and, for example, an ultrasound attenuating material such as ferrite or ceramics is added as necessary.

The flexible printed wiring board 31 is connected to the ultrasound oscillator 34 and the wiring cables inside the endoscope insertion portion 21, and electrically connects the ultrasound oscillator 34 to the ultrasound processor device 12.

The acoustic lens 5 converges ultrasound waves generated by the ultrasound oscillator 34 toward the observation target in the subject. The acoustic lens 5 is formed of, for example, a silicone resin (millable-type silicone rubber (HTV rubber), liquid silicone rubber (RTV rubber), and the like), a butadiene-based resin, or a polyurethane-based resin, and powders such as titanium oxide, alumina, or silica can be mixed as necessary.

The gas barrier layer 4 is formed from, for example, any of polyimide, polyethylene terephthalate, and parylene, and has gas-impermeable properties. The gas barrier layer 4 is disposed between the acoustic lens 5 and the piezoelectric element 3, and protects the piezoelectric element 3 from a gas permeating through the acoustic lens 5 from outside the ultrasound probe 1.

Typically, with respect to the ultrasound probe that is inserted into the subject, sterilization using a gas such as an ethylene oxide gas or a hydrogen peroxide gas plasma is often performed after use for observation. In this case, the gas used for sterilization may permeate through the acoustic lens 5 and reach the piezoelectric element 3, causing the piezoelectric element 3 to deteriorate. Because of the deterioration, the piezoelectric element 3 cannot sufficiently transmit and receive ultrasound waves in a desired frequency band, and, as a result, problems such as a decrease in resolution and sensitivity of the piezoelectric element may occur.

Since the gas barrier layer 4 prevents the gas used for sterilization from reaching the piezoelectric element 3, it is possible to suppress deterioration of the piezoelectric element 3 caused by the gas used for sterilization. Additionally, the acoustic matching layer 35 may be bonded to the ultrasound oscillator 34 via, for example, an adhesive. The gas used for sterilization may deteriorate the adhesive. Since the gas barrier layer 4 is disposed between the acoustic matching layer 35 and the acoustic lens 5, the gas barrier layer 4 also serves to protect the adhesive of the acoustic matching layer 35 from the gas used for sterilization.

In addition, from the viewpoint of blocking the gas used for sterilization, it is preferable that, in the radial direction DR of the outer peripheral surface 2A of the support member 2, the gas barrier layer 4 is thinner than the acoustic lens 5, and that the ratio of the thickness of the gas barrier layer 4 in the radial direction DR to the thickness of the acoustic lens 5 in the radial direction DR is 1/15 or less.

Further, in order to more reliably protect the plurality of piezoelectric elements 3 from the gas used for sterilization, it is preferable that the permeation rate of the gas such as a hydrogen peroxide gas or an ethylene oxide gas in the gas barrier layer 4 is lower than the permeation rate of the gas such as a hydrogen peroxide gas or an ethylene oxide gas in the acoustic lens 5, and is, for example, 20 g/(m²·day) or less. Note that [g/(m²·day)] is a unit representing the mass of gas per square meter that permeates through a target object such as the gas barrier layer 4 in one day.

Furthermore, in order to reliably protect the plurality of piezoelectric elements 3 with the gas barrier layer 4, it is preferable that, in a direction along the center axis of the cylindrical shape of the support member 2, the gas barrier layer 4 has a dimension longer than the plurality of piezoelectric elements 3, and that the plurality of piezoelectric elements 3 are located inside the gas barrier layer 4.

Additionally, it is preferable that the acoustic impedance of the gas barrier layer 4 is smaller than the acoustic impedance of the plurality of piezoelectric elements 3 and greater than the acoustic impedance of the acoustic lens 5. By setting the acoustic impedance of the gas barrier layer 4 in this way, the acoustic influence of the gas barrier layer 4 in the ultrasound probe 1 can be reduced.

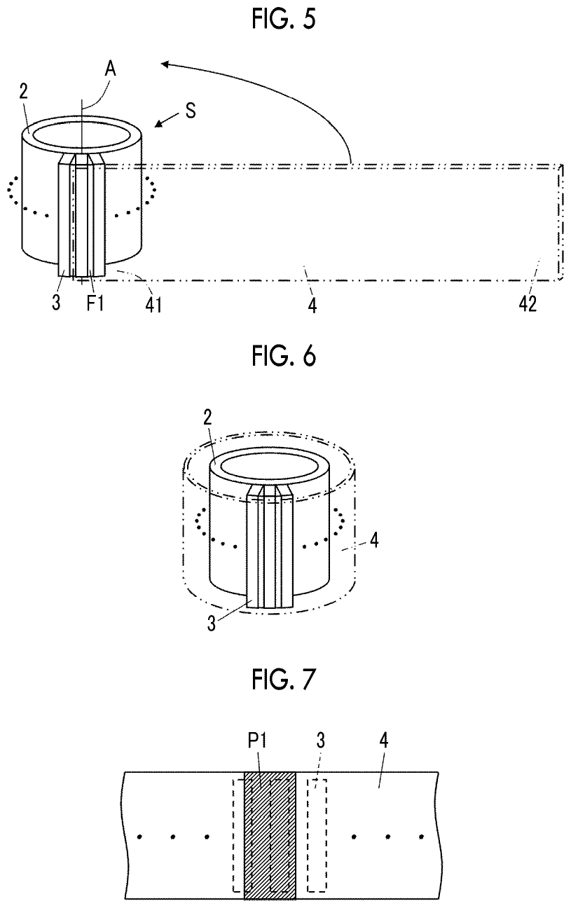

The gas barrier layer 4 is, for example, a film and can have a rectangular sheet shape as shown in FIG. 5. In a state in which one end portion 41 of the rectangular shape of the gas barrier layer 4 in the longitudinal direction is bonded to the piezoelectric element 3, the other end portion 42 of the rectangular shape of the gas barrier layer 4 in the longitudinal direction is rotated around a center axis A of the support member 2 having a cylindrical shape, and the gas barrier layer 4 is wound around the entire circumference of a structure S consisting of the support member 2, the plurality of piezoelectric elements 3, and the resin F1 that fills a space between the plurality of piezoelectric elements 3, whereby, as shown in FIG. 6, the gas barrier layer 4 can be disposed so as to surround the plurality of piezoelectric elements 3.

In this manner, by winding the gas barrier layer 4 around the plurality of piezoelectric elements 3 while, in the circumferential direction DC, overlapping both end portions in an arrangement direction of the plurality of piezoelectric elements 3, that is, in a direction of rotation about the center axis A, for example, the one end portion 41 and the other end portion 42 of the gas barrier layer 4 can be bonded together in a prescribed connection region P1, as shown in FIG. 7. In the example of FIG. 7, the overlapping portion in the connection region P1 overlaps the piezoelectric element 3.

In a portion where the overlapping portion in the connection region P1 overlaps the piezoelectric element 3, the acoustic impedance is different by an amount corresponding to one layer of the gas barrier layer as compared with a portion where the overlapping portion does not overlap the piezoelectric element 3; therefore, the frequency band of ultrasound waves that can be transmitted and received may be different.

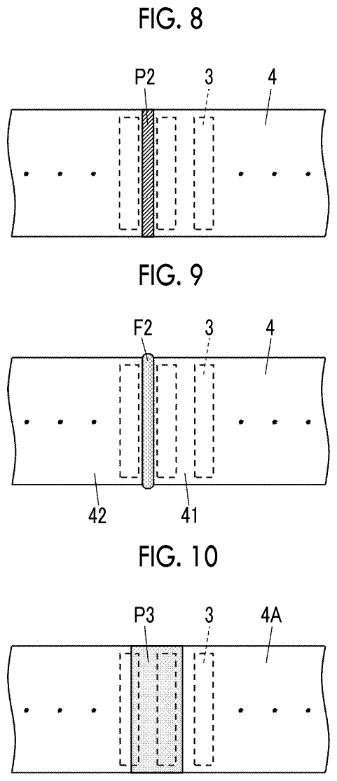

Therefore, for example, as shown in FIG. 8, an overlapping portion in a connection region P2 can be positioned between a pair of piezoelectric elements 3 that are adjacent to each other among the plurality of piezoelectric elements 3. In this case, since the overlapping portion does not overlap the piezoelectric element 3, the influence of the connection region P2 on the frequency band of ultrasound waves can be made slight.

In addition, for example, as shown in FIG. 9, the gas barrier layer 4 can be wound around the plurality of piezoelectric elements 3, with a gap formed between both end portions of the gas barrier layer 4 in the arrangement direction of the plurality of piezoelectric elements 3, and the gap can be filled with, for example, a resin F2 (adhesive) such as a polyimide resin. That is, by means of the resin F2, both end portions of the gas barrier layer 4 have the connection region P2. In this case as well, similarly to a case where the overlapping portion in the connection region P2 as shown in FIG. 8 is located between the pair of piezoelectric elements 3, the influence of the connection region P2 on the frequency band of ultrasound waves can be made slight.

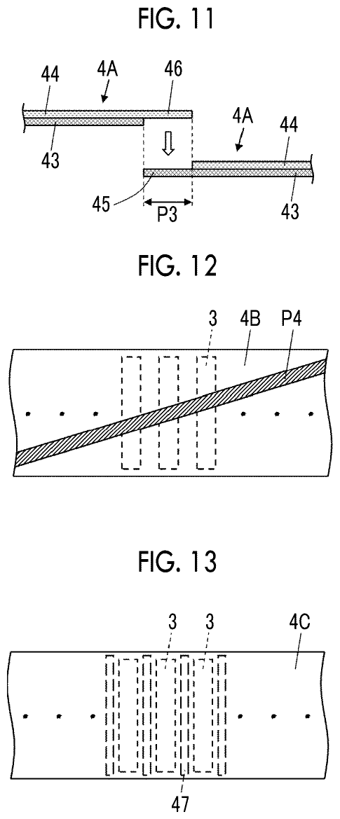

Further, it is also possible to use a gas barrier layer 4 in which both end portions are thinner than other portions. As shown in FIG. 10, an overlapping portion in a connection region P3 can be formed by bonding both end portions of such a gas barrier layer 4A together. The gas barrier layer 4A is configured by, for example, laminating two layers, namely a first layer 43 and a second layer 44, as shown in FIG. 11. In one end portion 45 of the gas barrier layer 4A, the second layer 44 is removed so that only the first layer 43 is present, and in the other end portion 46, the first layer 43 is removed so that only the second layer 44 is present. In the overlapping portion, the first layer 43 of the one end portion 45 and the second layer 44 of the other end portion 46 are bonded together.

Since the thickness of the overlapping portion in the connection region P3 is equivalent to the thickness of other portions, that is, portions of the gas barrier layer 4 that are not overlapped by the one end portion 45 and the other end portion 46, the frequency band of ultrasound waves that can be transmitted and received by the plurality of piezoelectric elements 3 can be made constant. Note that, although an example has been shown in which the overlapping portion in the connection region P3 overlaps the piezoelectric element 3, the overlapping portion can also be located between a pair of piezoelectric elements 3 that are adjacent to each other.

Additionally, for example, as shown in FIG. 12, a gas barrier layer 4B can also be wound around the structure S including the support member 2 and the plurality of piezoelectric elements 3 so as to form an overlapping portion in a connection region P4 that extends obliquely with respect to the center axis A of the support member 2. This overlapping portion extends so as to pass through all positions of the plurality of piezoelectric elements 3. In this way, since the overlapping portion overlaps approximately the identical area for each of the plurality of piezoelectric elements 3, it is possible to make the frequency band of ultrasound waves that can be transmitted and received by the plurality of piezoelectric elements 3 constant.

Further, as shown in FIG. 13, a gas barrier layer 4C including a plurality of protrusions 47 formed on the surface facing the plurality of piezoelectric elements 3 and having a width narrower than an interval between a pair of piezoelectric elements 3 adjacent to each other among the plurality of piezoelectric elements 3 can also be used. By using the protrusions 47 as marks in a case of winding the gas barrier layer 4C around the structure S including the support member 2 and the plurality of piezoelectric elements 3, the gas barrier layer 4C can be accurately wound along the arrangement direction of the plurality of piezoelectric elements 3. In a case where the gas barrier layer 4C is wound around the structure S in a state of being tilted with respect to the arrangement direction of the plurality of piezoelectric elements 3, both end portions of the gas barrier layer 4C are bonded together in the overlapping portion of the gas barrier layer 4C in a state of being offset from each other in the direction along the center axis A of the support member 2. In this case, the piezoelectric element 3 may not be sufficiently protected from the gas used for sterilization.

In addition, by using the protrusions 47 as marks in a case of winding the gas barrier layer 4C around the structure S including the support member 2 and the plurality of piezoelectric elements 3, it is also possible to accurately align both end portions of the gas barrier layer 4C in the arrangement direction of the plurality of piezoelectric elements 3. As a result, for example, as shown in FIG. 8, the overlapping portion in the connection region P2 can be accurately disposed between the piezoelectric elements 3, and, as shown in FIG. 9, both end portions of the gas barrier layer 4C can also be accurately disposed between the piezoelectric elements 3.

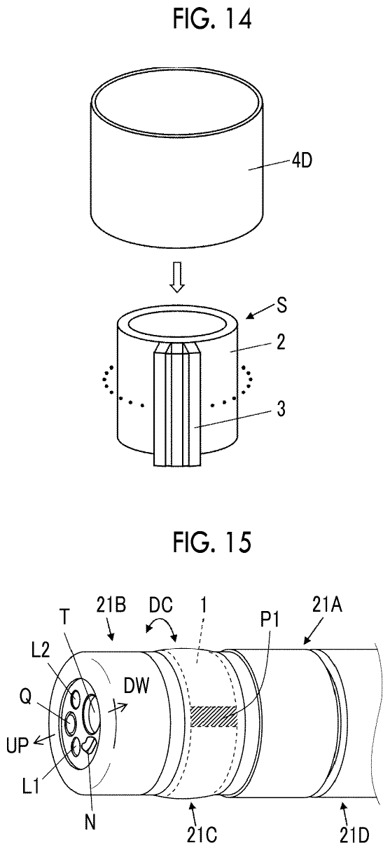

Further, for example, as shown in FIG. 14, a gas barrier layer 4D having a seamless tubular shape can also be used. The gas barrier layer 4D can be bonded, by a resin such as a silicone resin, to the structure S, for example, in a state of being disposed at a position at which the structure S including the support member 2 and the plurality of piezoelectric elements 3 is surrounded. In this case, the filling of the resin F1 between the plurality of piezoelectric elements 3 can be performed before bonding between the gas barrier layer 4D and the structure S, and can also be performed simultaneously with bonding between the gas barrier layer 4D and the structure S.

Additionally, in a case where the gas barrier layer 4D having a seamless tubular shape is formed of, for example, a material that contracts elastically or by heating, the gas barrier layer 4D can be fixed to the structure S including the support member 2 and the plurality of piezoelectric elements 3 by, for example, contracting the gas barrier layer 4D in a state of being disposed at a position at which the structure S is surrounded. In this case, the filling of the resin F1 between the plurality of piezoelectric elements 3 can be performed before fixing the gas barrier layer 4D to the structure S.

Since the gas barrier layer 4D has no seam, it is possible to make the frequency band of ultrasound waves that can be transmitted and received by the plurality of piezoelectric elements 3 constant.

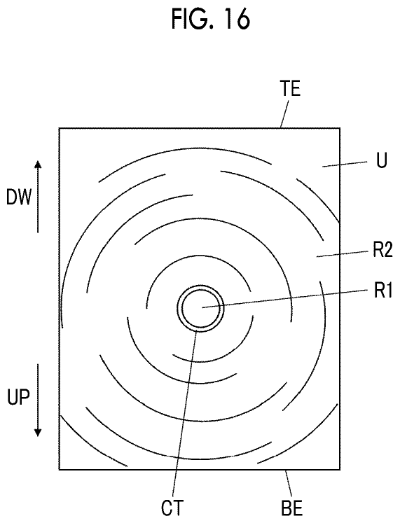

Further, in a case where the gas barrier layer 4 has the connection region P1, for example, the connection region P1 can be localized, as shown in FIG. 15, at a prescribed specific position in the circumferential direction of the endoscope insertion portion 21, that is, in the circumferential direction DC of the outer peripheral surface 2A of the support member 2 shown in FIG. 3. In this case, for example, the connection region P1 can be disposed on a side opposite to the upward direction UP of the endoscope observation portion 21B, that is, on a side opposite to the optical observation window Q with respect to the center axis of the endoscope insertion portion 21. In this case, the connection region P1 can also be said to be disposed on the downward direction DW side with respect to the center axis of the endoscope insertion portion 21. The connection region P1 can also be said to be disposed on a treatment tool outlet port T side with respect to the center axis of the endoscope insertion portion 21.

Here, the reception signal generated by the ultrasound probe 1, which is of a radial type, is transmitted to the ultrasound processor device 12, and, for example, an ultrasound image U as shown in FIG. 16 is generated. The ultrasound image U includes an upper end portion TE, a lower end portion BE, a circular probe region R1 corresponding to the ultrasound probe 1 at a central part thereof, and a tomographic region R2 that is a region outside the probe region R1 and that represents a tomographic image in the subject. In the ultrasound image U, a direction from the upper end portion TE toward the lower end portion BE corresponds to the upward direction UP, and a direction from the lower end portion BE toward the upper end portion TE corresponds to the downward direction DW. A plurality of positions on a contour CT of the probe region R1 correspond to the plurality of piezoelectric elements 3 of the ultrasound probe 1.

In the connection region P1 of the gas barrier layer 4 in the ultrasound probe 1, since both end portions of the gas barrier layer 4 are overlapped with each other, the acoustic impedance is different by an amount corresponding to one layer of the gas barrier layer 4 as compared with a region in which both end portions of the gas barrier layer 4 are not overlapped with each other. Therefore, for ultrasound waves that pass through the connection region P1, the frequency band that can be transmitted and received by the ultrasound oscillator 34 may be different as compared with the frequency band for ultrasound waves that pass through regions other than the connection region P1. Accordingly, in a case where the ultrasound image U is acquired using the ultrasound probe 1 in which the connection region P1 is present, resolution of portions in the ultrasound image U that correspond to the ultrasound oscillator 34 overlapping the connection region P1 and the ultrasound oscillator 34 located in the vicinity of the connection region P1 may deteriorate as compared with the original resolution.

Meanwhile, typically, the user such as a physician often acquires the endoscope image and the ultrasound image U in a state in which an upward direction UP side of the distal end portion 21A of the endoscope insertion portion 21 is pressed against the inside of the body cavity of the subject. Therefore, the user such as a physician often performs diagnosis mainly by checking a region on the upward direction UP side, that is, a lower side, relative to the probe region R1 in the ultrasound image U, whereas a region on an upper side relative to the probe region R1 in the ultrasound image U is often not used for diagnosis.

Since the connection region P1 is disposed in a direction opposite to an optical observation window Q side with respect to the center axis of the endoscope insertion portion 21, that is, in a direction opposite to the upward direction UP with respect to the center axis, that is, it is disposed, in the circumferential direction DC, at a position on the contour CT of the probe region R1 in the ultrasound image U on an upper end portion TE side, only the region on the upper side relative to the probe region R1 in the ultrasound image U is affected by the connection region P1. That is, the region on the lower side relative to the probe region R1 in the ultrasound image U, which is often used by the user such as a physician for diagnosis, is not affected by the connection region P1 and can have sufficient resolution.

In this way, with the ultrasonic endoscope 11 and the ultrasound diagnostic system in the embodiment of the present invention, it is possible to acquire the ultrasound image U that is useful for diagnosis while suppressing deterioration of the piezoelectric elements caused by the gas used for sterilization.

Although an example has been described in which the ultrasound probe 1 of the embodiment of the present invention is mounted on the ultrasonic endoscope, the present invention is not particularly limited to this. The ultrasound probe 1 can also be mounted on, for example, an observation device that is inserted into a rectum or the like of the subject in order to capture an ultrasound image of the rectum of the subject and that does not include an endoscope.

In addition, although an example has been described in which the gas barrier layer 4 is a film, the gas barrier layer 4 can also be formed by, for example, vapor-depositing onto the plurality of piezoelectric elements 3, applying a fluid material of the gas barrier layer 4 to the plurality of piezoelectric elements 3 and then curing the fluid material, or dipping the structure S into the fluid material of the gas barrier layer 4 and then curing the fluid material.

Further, the connection regions P1, P2, P3, and P4 of the gas barrier layer 4 can have, for example, in the circumferential direction DC, a width dimension corresponding to an angular range greater than 0 degrees and equal to or less than 30 degrees about the center axis A of the cylindrical shape of the support member 2, that is, a width dimension equal to or less than 30 degrees/360 degrees= 1/12 of the entire circumference in the circumferential direction DC. As a result, it is possible to further reduce an influence of the connection regions P1, P2, P3, and P4 on the ultrasound image U while suppressing deterioration of the piezoelectric elements caused by the gas used for sterilization.

Furthermore, although it has been described that the ultrasound observation portion 21C is located on the bending portion 21D side relative to the endoscope observation portion 21B, the ultrasonic endoscope 11 may have a configuration in which the endoscope observation portion 21B is located on the bending portion 21D side relative to the ultrasound observation portion 21C.

Additionally, although it has been described that the cleaning nozzle N is disposed between the first illumination window L1 and the treatment tool outlet port T, the position of the cleaning nozzle N is not particularly limited as long as the optical observation window Q, the first illumination window L1, and the second illumination window L2 can be cleaned, and it may be disposed, for example, between the first illumination window L1 and the optical observation window Q, or between the second illumination window L2 and the optical observation window Q.

Moreover, although it has been described that, regarding the upward direction UP, a direction in which the bending portion 21D of the endoscope insertion portion 21 bends, a direction that the optical observation window Q faces with respect to the center axis of the endoscope insertion portion 21, and a direction from the upper end toward the lower end in the ultrasound image generated in the ultrasound observation portion 21C are equal to one another, the direction in which the bending portion 21D of the endoscope insertion portion 21 bends, the direction that the optical observation window Q faces with respect to the center axis of the endoscope insertion portion 21, and the direction from the upper end toward the lower end in the ultrasound image generated in the ultrasound observation portion 21C may be different from one another depending on specifications and the like of the ultrasound diagnostic system 10. In such a case, it is preferable that the connection regions P1, P2, P3, and P4 are disposed at least at a position corresponding to the region on the upper side relative to the probe region R1 in the ultrasound image U.

### EXPLANATION OF REFERENCES

1: ultrasound probe
2: support member
2A: outer peripheral surface
3: piezoelectric element
4, 4A, 4B, 4C, 4D: gas barrier layer
5: acoustic lens
10: ultrasound diagnostic system
11: ultrasonic endoscope
12: ultrasound processor device
13: endoscope processor device
14: light source device
15: monitor
16: water supply tank
17: suction pump
18A: ultrasound connector
18B: endoscope connector
18C: light source connector
19A: air/water supply tube
19B: suction tube
21: endoscope insertion portion
21A: distal end portion
21B: endoscope observation portion
21C: ultrasound observation portion
21D: bending portion
21E: flexible portion
22: operation portion
22A: air/water supply button
22B: suction button
22C: angulation knob
22D: treatment tool insertion port
23: universal cord
31: flexible printed wiring board
32: backing material layer
33: dematching layer
34: ultrasound oscillator
35: acoustic matching layer
41, 45: one end portion
42, 46: the other end portion
43: first layer
44: second layer
47: protrusion
A: center axis
BE: lower end portion
CT: contour
DC: circumferential direction
DR: radial direction
DW: downward direction
F1, F2: resin
L1: first illumination window
L2: second illumination window
N: cleaning nozzle
R1: probe region
R2: tomographic region
P1, P2, P3, P4: connection region
Q: optical observation window
S: structure
T: treatment tool outlet port
TE: upper end portion
U: ultrasound image
UP: upward direction

What is claimed is:

1. An ultrasound probe comprising:
a support member that includes an outer peripheral surface having a cylindrical shape;
a plurality of piezoelectric elements arranged on the outer peripheral surface in a circumferential direction of the outer peripheral surface;
an acoustic lens that is disposed outside the plurality of piezoelectric elements in a radial direction of the outer peripheral surface and that covers the plurality of piezoelectric elements; and
a gas barrier layer that is disposed between the plurality of piezoelectric elements and the acoustic lens,
wherein a permeation rate of a hydrogen peroxide gas or an ethylene oxide gas in the gas barrier layer is lower than a permeation rate of the hydrogen peroxide gas or the ethylene oxide gas in the acoustic lens, and
in the radial direction, the gas barrier layer is thinner than the acoustic lens.

2. The ultrasound probe according to claim 1,
wherein a ratio of a thickness of the gas barrier layer in the radial direction to a thickness of the acoustic lens in the radial direction is 1/15 or less.

3. The ultrasound probe according to claim 1,
wherein the piezoelectric element includes an acoustic matching layer, and
the gas barrier layer is disposed between the acoustic matching layer and the acoustic lens.

4. The ultrasound probe according to claim 1,
wherein the plurality of piezoelectric elements are located inside the gas barrier layer in a direction along a center axis of the cylindrical shape of the support member.

5. The ultrasound probe according to claim 1,
wherein an acoustic impedance of the gas barrier layer is smaller than an acoustic impedance of the piezoelectric element and is greater than an acoustic impedance of the acoustic lens.

6. The ultrasound probe according to claim 1,
wherein the permeation rate of the hydrogen peroxide gas in the gas barrier layer is 20 g/(m²·day) or less.

7. The ultrasound probe according to claim 1,
wherein the gas barrier layer is a film wound around the plurality of piezoelectric elements.

8. The ultrasound probe according to claim 7,
wherein the gas barrier layer includes a connection region in which an adhesive that fills a space between both end portions of the gas barrier layer in the circumferential direction is located, or in which the gas barrier layer is overlapped in the circumferential direction.

9. The ultrasound probe according to claim 8,
wherein the connection region has, in the circumferential direction, a width dimension corresponding to an angular range greater than 0 degrees and equal to or less than 30 degrees about a center axis of the cylindrical shape of the support member.

10. The ultrasound probe according to claim 8,
wherein an overlapping portion of the gas barrier layer overlaps any of the plurality of piezoelectric elements.

11. The ultrasound probe according to claim 8,
wherein an overlapping portion of the gas barrier layer is located between a pair of piezoelectric elements adjacent to each other among the plurality of piezoelectric elements.

12. The ultrasound probe according to claim 8,
wherein the gas barrier layer is wound around the plurality of piezoelectric elements, with a gap formed in the connection region between both end portions of the gas barrier layer in an arrangement direction of the plurality of piezoelectric elements, and the gap is filled with a resin.

13. The ultrasound probe according to claim 8,
wherein the gas barrier layer includes a plurality of protrusions formed on a surface facing the plurality of piezoelectric elements and having a width narrower than an interval between a pair of piezoelectric elements adjacent to each other among the plurality of piezoelectric elements.

14. The ultrasound probe according to claim 7,
wherein the gas barrier layer has a seamless tubular shape.

15. An ultrasonic endoscope comprising:
an endoscope insertion portion that is inserted into a body cavity of a subject and that extends in an axial direction; and
an endoscope observation portion and an ultrasound observation portion that are disposed at a distal end of the endoscope insertion portion,
wherein the ultrasound observation portion includes the ultrasound probe according to claim 1.

16. The ultrasonic endoscope according to claim 15,
wherein the gas barrier layer is a film wound around the plurality of piezoelectric elements along the circumferential direction of the outer peripheral surface, and has both end portions disposed together within a prescribed connection region, and
the connection region of the gas barrier layer is localized at a prescribed position in the circumferential direction of the outer peripheral surface.

17. The ultrasonic endoscope according to claim 16,
wherein the endoscope observation portion includes an optical observation window that, at a position eccentric with respect to a center axis of the endoscope insertion portion, is directed forward from the distal end of the endoscope insertion portion along the axial direction of the endoscope insertion portion, and
the connection region of the gas barrier layer is disposed on a side opposite to the optical observation window with respect to the center axis of the endoscope insertion portion.

18. The ultrasonic endoscope according to claim 16, further comprising:
a treatment tool that is inserted through the endoscope insertion portion,
wherein the endoscope observation portion includes a treatment tool outlet port that, at a position eccentric with respect to a center axis of the endoscope insertion portion, is directed forward from the distal end of the endoscope insertion portion along the axial direction of the endoscope insertion portion and through which the treatment tool is led out toward an inside of the subject, and
the connection region of the gas barrier layer is disposed on a treatment tool outlet port side with respect to the center axis of the endoscope insertion portion.

19. The ultrasonic endoscope according to claim 16, further comprising:
an operation portion that is used by a user to operate the endoscope insertion portion,
wherein the endoscope insertion portion includes a bending portion that bends in a prescribed upward direction in response to an operation of the user via the operation portion and in a downward direction opposite to the upward direction, and
the connection region of the gas barrier layer is disposed on a downward direction side with respect to a center axis of the endoscope insertion portion.

20. An ultrasound diagnostic system comprising:
the ultrasonic endoscope according to claim 16; and
a processor connected to the ultrasonic endoscope,
wherein the processor is configured to generate an ultrasound image based on a reception signal obtained by transmitting and receiving ultrasound waves toward a subject via the ultrasound observation portion,
the ultrasound image has an upper end portion, a lower end portion, a probe region corresponding to the ultrasound probe, and a tomographic region outside the probe region,
a plurality of positions on a contour of the probe region correspond to the plurality of piezoelectric elements of the ultrasound probe, and
the connection region of the gas barrier layer is disposed, in the circumferential direction of the outer peripheral surface, at a position on the contour of the probe region in the ultrasound image that corresponds to a position on an upper end portion side.
